(19) United States
(12) Patent Application Publication
(10) Pub. No.: US 2015/0190468 A1
(43) Pub. Date: Jul. 9, 2015

(54) Methods for Creating and Using Platelet Extracts

(71) Applicant: Mark Roedersheimer, Denver, CO (US)

(72) Inventor: Mark Roedersheimer, Denver, CO (US)

(21) Appl. No.: 14/148,041

(22) Filed: Jan. 6, 2014

(51) Int. Cl. A61K 38/18; A61K 35/14

(52) U.S. Cl. CPC A61K 38/1858; A61K 35/19

(57) ABSTRACT

This invention provides processes for creating and stabilizing extracts of platelets possessing improved properties for tissue engineering and cell culture applications relative to other methods, that can be made without the use of freeze lysis, the resulting compositions, and methods for using them.

### RELATED APPLICATIONS

The present application claims benefit of U.S. Provisional Patent Application No. 61/748,807, filed Jan. 4, 2013, which herein is incorporated by reference in its entirety.

### STATEMENT REGARDING FEDERALLY RESEARCH

This invention was made without any government funding support.

### STATEMENT REGARDING JOINT RESEARCH AGREEMENT

No research agreements exist between the inventor and other entities

### FIELD OF THE INVENTION

The present invention relates to improved methods of collecting, stabilizing and applying mixtures of growth factors from platelets.

### BACKGROUND OF THE INVENTION

U.S. Pat. No. 8,282,963 (hereinafter, the “'963 patent”), the entire disclosure of which is incorporated by 33780-0000 reference herein, discloses mixtures with enhanced angiogenic activity for certain therapeutic applications, but might be limited for broader applications by creating an imbalance of proangiogenic factors and antiangiogenic factors and other elements naturally present in platelets. Considering the “cancering” concepts of Agus and Hillis and compelling research by Folkman's group (Angiogenesis. 2012 June; 15(2):265-73., Blood. 2010 Jun. 3; 115(22):4605-13 Am J. Hematol. 2010 July; 85(7):487-93., Blood. 2009 Mar. 19; 113(12):2835-42, Blood. 2008 Feb. 1; 111(3):1201-7., Blood. 2008 Feb. 1; 111(3):1227-33.,), and others (Blood. 2011 Apr. 7; 117(14):3907-11, Blood. 2011 Apr. 7; 117(14):3893-902., Platelets. 2010; 21(2):85-93., Clin Cancer Res. 2008 May 15; 14(10):3070-6. PNAS. USA. 2005 Jan. 4; 102(1):216-20.), these “unbalanced” mixtures may favor “cancering” responses when applied inappropriately. A benefit is that the presently described processes can create compositions less likely to result in cancer in patients who might be treated inappropriately with products generated by methods described in the '963 patent. The present compositions also may have greater therapeutic value in supporting “non-angiogenic” needs of wound healing processes. For example, the compositions may provide more balanced nourishment for growth of human cells than mixtures depleted of low MW factors. These effects can be studied in cell culture.

The conceptual foundation of the processes disclosed herein has been informed by knowledge of signaling imbalances involved in generally recognized angiogenesis-dependent disease processes, such as cancer, pulmonary hypertension and preeclampsia, among many others. Conditions of insufficient angiogenesis, such as heart disease and lower extremity ischemic conditions also have informed these approaches.

Other aspects were informed by the concept of a breakdown in the homogeneous equilibrium condition, perhaps first significantly detailed by Alan Turing (Philosophical Transactions of the Royal Society (part If), Vol. 237, pp. 37-72 (1953)). What we consider to be the best conceptual framework to explain the emergence of biological patterns seems to have been replaced by an overly reductive, mechanistic viewpoint and packaged into the idea of “Chaos” or “Chaos Theory.” The underlying idea is simple, hence its power. A homogeneous state can interact with any weak influence, like a field such as gravity, to then evolve into a state of higher order via mechanisms of the reaction-diffusion theory (Science. 1994 Apr. 8; 264(5156):245-8). The idea probably has power to explain how a flower opens or a pregnant woman goes into labor. It is like an egalitarian rather than hierarchical, or “top-down” control theory. While Chaos Theory and fractal models yield deterministic results, simply dependent on initial conditions, the R-D concept can be seen as “non-deterministic,” intrinsically adaptive, and open-ended in terms of processes it can describe. It does not lend itself to representation in any mathematically final or “absolute” manner. Excellent descriptions of R-D system models exist in Wikipedia online at the time of this filing.

This work has been informed by the idea that any biological network of elements (neurons, immune cells, any network of cells in the body, or mixtures of biomolecules alone) selected by evolution, has an intrinsic capacity to attach meaning to perceived “invariant” aspects in the system under stress. Any number of invariants, which can be nested within one another, act as a basis for image formation, which those skilled in the art can understood to exist outside visual images, into any realm of perception such as gustatory and olfactory, the immune system, or the dynamics underlying the functioning and metabolism of any tissue in the body. This idea of a network internalizing information from the environment to inform categorical decision making could be seen, by those skilled in the art, to be in line with Alan Turing's work leading to the building of a system of indexed memory, that led to modern computers, to aid in the identification of U-boat locations during WWII, and is now being recognized as a reasonable basis for decision making in the brain (Gold/Shadlen, Neuron, vol 36, no 2, 10 Oct. 2002, pg 299-308). The idea is represented by the circumstance of an infant, upon achieving the neurologic capacity of “object permanence,” to make an inversion on a perceived symmetry represented by the problem of a bottle presented to her backwards. One skilled in the art can recognize that this is essentially the same as perceiving a key set of symmetries in a chess game from which one opponent can effectively find a leverage point upon which to “invert” the dynamic, and thereby gain an advantage. However, a chess game is represented in a finite number of dimensions, while true biological systems must work puzzle solving under conditions of unbounded dimensionality in order to survive.

This work has been informed by the idea that memory, and action properties can be displayed from mixtures of biomolecules isolated from human tissue, such as those in platelet extracts, or bone, when delivered in vivo (Vasc. Cell 2011 Feb. 2; 3(1):4.; Angiogenesis. 2005; 8(4):327-38. Epub 2006 Jan. 7.) The evidence presented here can be interpreted to show “learning” or “training” capability of such natural mixtures. A paper by Tabony (Science. 1994 Apr. 8; 264(5156):245-8) describes how mixtures of reacting and diffusing tubulin and GTP, with a GTP regenerating system, can display a morphological bifurcation in the final gelation pattern which is sensitive to the orientation of gravity. This can be seen as a form of non-local memory, which is “indexable” simply through the influence of an external field. Likewise, the influence of external factors, such as infiltration of acidity, other extraction materials, sieving properties of clots, or temperature, can be seen as influencing the final bioactivity of these mixtures by a similar mechanism, suggesting a capability to “perceive” the environment, and leading to altered capacity for action, through incorporation of sensed information that transforms the network architecture of potential interactions between the molecules in subtle ways. These changes are probably interpreted most effectively at this time, in altered bioactivity. Those skilled in the art would claim there is a clear physical basis for these alterations, it is just that the means to understand them is easiest using bioassays, rather than physical analysis of components, since the physical alterations will have affected such a large numbers of elements in the mix, in ways subtle enough to make it impractical (with current means) to understand them on a purely compositional (i.e. reductive) basis.

These inventions have been informed by the idea that another form of memory must exist in multicellular organisms, which is not strictly referable to DNA alone, and may be present in the organized complexity of biomolecules and chemical species of any type, in the organism. The concept of a Hilbert Space, expanded beyond the realms typically found in engineering, physics and computer science, areas largely ruled by reductive paradigms, and frankly overly mechanistic views, needs be accommodated for the fullest appreciation of what is revealed. Extensive exposure to modeling techniques with ordinary and partial differential equations, as well as Tensor methods, have informed these ideas.

This work seeks to provide a counterbalance to the idea that “memory,” can be stored strictly in localized material forms, such as a DNA molecule, or at anatomically defined sites in the brain. This work has been informed by the insight that natural, or biological memory systems are perhaps well described as cryptanalysis systems designed to work with a two-key or public-key mechanism, in which the second key, is the perceived environment. A projectile entering our visual space has to be recognized rapidly and accurately for its ability to harm us, and comparison to a stored memory to make the determination seems untenable. Similarly, molecular signatures of infectious organisms intercepting the perceptual space of our immune system also must be interpreted, in a timely manner, for their ability to harm us.

Where or how a “recognition memory” is stored in our immune system appears to be unknown. To employ a simple example, how do we typically remember where we left our car keys, if lost from the night before? Perhaps we decrypt our memory of their location by looking around our homes, where we are able to receive environmental information remaining from the previous day that provides a second key. We would claim that memory deficits or inability to retrieve information, referable to lesions in the nervous system are due to disturbance of the neural network architecture that supports the cryptanalysis, rather than the ablation of specific “memory storage” sites. Anomalies of immune system function can also be conceived as resulting from specific alterations undermining optimal cryptanalytic functioning of the network architecture necessary for successful clearance of a pathogen or circumstances leading to autoimmune diseases.

This work has been informed by the realization that angiogenic response must be stimulated in the body in a non-reductive, non-deterministic manner, i.e. not referable to defined signals, and using some form of “encryption” in the distribution of signaling molecules, contained in large part within factors such as those in platelets. This is attributed to microorganisms having the power to evolve blockers and modulators of these signals, suggesting that the human body must be changing constantly and evolving the encryption scheme. The concept of the Red Queen of van Valen has informed these inventions.

These innovations have bee informed by the idea that probability models that make assumptions about random processes should be cautiously considered in biological systems research efforts. Any random process would have metabolic costs, but no clear benefits to the organism, when carefully examined. The signals in the organism that currently are un-interpretable and that might be lumped into the category of “noise” are likely important, although perhaps incapable of our understanding. These approaches might have been influenced by relaxing our requirement to find an absolute truth.

This work has been influenced by the field of information theory and, in part, is an effort to expand that field into the realm of biological information systems.

This work has been informed by the realization that as much information can be present in waves of molecules of any size, such as proton concentration (acidity), other small molecules, fluctuations in pressure, temperature, shear, in the same measure as any product of a DNA molecule. It should be considered that the most widely recognized, and perhaps most ancient family of morphogens, the TGFbeta superfamily, consists of molecules that, like DNA, are robust and can stand up to harsh extraction conditions allowing for rather simple isolation and analysis. It is an assumption of this work that the current primacy of DNA and this family of morphogens is, in part, related to the fact that other aspects of biological complexity are easily lost or degraded during extraction and collection processes, and hence largely missed for their relevance. This work is informed by the idea that benefits can be found in focusing on the “whole” mixtures of factors, with attention to development of extraction processes that leave important, and often chemically labile, elements accounted for, present and able to manifest their natural influences in bioassays. This requires looking to types of art outside the realms traditionally defined as “molecular biology.” Traditional biochemistry may be a more appropriate description of these efforts.

These methods have been informed by study of diverse types of transformations of biological material into forms suited for healthy human consumption, particularly those elemental to human survival over eons, such as zymurgy and the handling of dairy products (cheese/yogurt making) Making a chocolate mousse, in this sense, includes bioprocess engineering that involves respecting the wholeness in each element in just the correct way to allow a sequential integration that results in an ideal finished result.

In some aspects, these ideas have been informed by what appears to be a broadened concept of a Monte Carlo simulation, particularly that of a “simulated annealing”—a Monte Carlo methodology as pertains to decoding, or “cryptanalysis” in biological systems. We would claim this long has been known to artists in the animal husbandry, gardening, culinary and zymurgical field who can find meaningful solutions with the method, in areas where no simple, or “closed-form expression” (i.e. a discrete, unique solution), is capable of existing.

“Monte Carlo methods vary, but tend to follow the following pattern:

1. Define a domain of possible inputs.

2. Generate inputs randomly from a probability distribution over the domain.

3. Perform a deterministic computation on the inputs.

4. Aggregate the results.” (Quoted from Monte Carlo Method Wikipedia pg, on Jan. 5, 2014)

A modification of this algorithm for optimization of biological systems research, and design problems has informed the innovations revealed here, along these lines:

1. From our work with platelet mixtures, the “domain of possible inputs” would be mixtures that can result from processes applied to the extraction and collection of factors from platelets. This can be generalized to any biomaterial, and applied processes for their extraction.

2. Generation of inputs, for our case, would be via the choice of processes applied to the platelets, but this has not been random. It has been guided by expertise in other areas of extraction of botanicals such as malt, tea, coffee, and expertise in crafting distinct effects, such as flavors, in culinary and zymurgical efforts. The key point is to search for a circumstance in other areas of bioprocess design (the culinary/zymurgical realm is most rich) and translate that paradigm to the question at hand which one is seeking to optimize or solve.

3) The “deterministic computation on the inputs” has been done, primarily, but not exclusively, for this work on platelets in the past using the Matrigel plug assay. Basically, the determination is handled by the body of an animal, and the eye of the researcher counting capillaries in plug sections. This is generalizable to any “bioassay.” A better bioassay, that is more representative of the phenomena of interest, will give an “annealing” to an optimal solution more rapidly than a poor quality bioassay.

4) In reference to the first optimization of processes for collecting angiogenic activity from platelets in the '963 patent, aggregating the results allowed us to make rational choices about which process modification gave improved activity, that then allowed more rational choices about process modifications to influence favorably the “domain of possible inputs.”

In essence, this algorithm, which we believe has been used by gardeners, livestock managers, culinary and zymurgical artists for eons, amounts to a kind of “decryption” of the solution to a biological problem. For example, it was discovered that barley could be kept stable over the winter to help people survive by “converting” it to beer. Relatedly, cheese and yogurt were discovered to transform and stabilize milk, which is prone to sour. These were emergent, solutions, decrypted by nature and the mind of man in balance.

Those skilled in the art can see that decryption is aided by possession of a paired “plain text” message and the enciphered form of the message in hand. In the biomedical realm, the “enciphered message” is analogous to the set of signals in a particular wound environment known to be present, which can derive from lab tests on that patient and the literature on the particular disease process. The plaintext is a clinician's interpretation of what the body logically is attempting to accomplish at the site for a particular condition, such as, but not limited to, stimulating or inhibiting new blood vessel formation, regulating oxidative stress, controlling infectious risk, regulating complement activity, or modulation of pain. This understanding does not need to be perfect to allow the clinician to begin “attacking the traffic” and start decrypting the disease condition. Weighting of evidence as per the concept of a “ban”, referable to Turing, will likely hinge on these aspects, as detailed below.

In cryptanalysis multiple messages sent with the same key are said to be “in depth,” and it can offer an advantageous circumstance to cryptanalysis. In biomedical cryptanalysis, there could be many cases where this advantage can be revealed from looking at the wound environment from different perspectives, as guided by different bioassays.

Another concept that has informed these inventions is the idea that all diseases of the body can be conceived in terms of a breakdown between local and global encryption schemes, leading to failure of information and resources reaching the appropriate sites to favor optimal resolution of the disease condition. Building tools for the identification and correction of the breakdown to reestablish proper flows of information and resources in the body is but one goal of this work.

The '963 patent provides a method for collecting a mixture of growth factors from lysates of platelets that involves removal of low-molecular weight elements via a dialysis process. A product described has a dose-dependent angiogenic activity of greater activity and so it can be important for treatment of conditions in which a human or animal recipient lacks the ability to manifest sufficient angiogenic activity at a wound site for proper healing to naturally occur. Such is the case for ischemic heart and lower extremity disease, diabetic ulcers, metabolic infirmities of age and chronic disease, and seen in severely burned patients. Such applications can be described as “therapeutic angiogenesis” (Ther. Deliv. 2012 June; 3(6):693-714. Cardiol. Rev. 2008 July-August; 16(4):163-71). One explanation for how the product of the '963 patent leads to enhancement of angiogenic activity and wound healing potency is that the low molecular weight fraction (dialysate), which is excluded contains some factors that naturally inhibit angiogenic activity. At microvascular injury sites, which are recognized to occur routinely among those skilled in the art, and underlie internal bleeding that occurs in patients with low platelet counts, the clots that form normally around platelets adherent at such sites conceivably could perform the function of a “filter” or mesh that allows low weight elements to diffuse from the site into the blood, allowing higher weight factors to manifest enhanced angiogenic responses at the wound site. Thus, a product of the '963 patent may mimic a natural aspect of human biology to enhance the angiogenic activity of materials produced outside the body.

Healthy patients who have the ability to manifest a sufficient angiogenic response at a wound site, such as for surgical spinal fusion or topical healing of laser-resurfacing wounds, or any cosmetic procedure in a healthy patient such as dental perioplastic procedures, laser-resurfacing of wounds, or any disease condition needing correction in a person not suffering a metabolic abnormality, such as, but not limited to, diabetes, that could compromise angiogenic response, may be better-served by products from platelets that do not have a portion of the natural inhibitors removed. It seems reasonable to assume that nature put all of the factors into platelets for a reason. Evidence by Folkman's group and others mentioned above indicates that a wound, tumor microenvironment, or other factors in the patient's body can have a critical impact on the factors released by platelets, whether for good or ill effect. Thus, care should be taken in determining what factors to remove, and any exclusions or re-incorporations should be guided rationally by the specific imbalances of the wound that the clinical practitioner is seeking to correct. This goal underlies a purpose for the present invention.

The additions to the art provided by the present invention are important because cancer is a condition that is associated with excess or imbalanced angiogenic activity in tissues, akin to what may be an essential feature of the product disclosed in the '963 patent, and other proangiogenic therapies such as rhPDGF-BB, VEGF, and FGF. It is now known that rhPDGF-BB (REGRANEX®) dosed in excess (and apart from the antiangiogenic factors, natural PDGF is found alongside in platelets) is associated with an increased risk of cancering in humans. For similar reasons, placing too much of an unbalanced mixture, like that described in the '963 patent, into an internal surgical site, at which the ideal dose may be unknown, might be expected to increase the risk of cancer. Natural, optimal, healthy wound healing probably involves a separation in time between delivery of the pro-angiogenic and the anti-angiogenic factors, such that, early in healing, the agonist factors are delivered, and then, when the angiogenic response is contraindicated and to allow restoration of tissue quiescence, the antagonist factors become manifest. This is suggested in results reported by Italiano and Folkman (Blood. 2008 Feb. 1; 111(3):1227-33), demonstrating that platelet alpha granules are segregated between those containing proangiogenic factors and anti-angiogenic factors, and that the two populations selectively could be released via different receptor mechanisms. The point is that nature appears to favor a method of providing balance between these angiogenic regulatory elements for the optimal outcome. Dysregulation of these selective elaboration mechanisms probably has a role in cancer progression. It has been found that animals with occult (undetectable) tumors can show evidence of the angiogenic factor secreting tumor in the elevated levels of angiogenic factors in platelets, which selectively concentrate these factors in to them up steep gradients from the blood (Blood. 2009 Mar. 19; 113(12):2835-42), perhaps explaining the unreliability of attempts to detect the early presence of cancer by analysis of serum/plasma for angiogenic factor levels (Am J Hematol. 2010 July; 85(7):487-93., Angiogenesis, 2012 June; 15(2):265-73). The platelets appear to be a key control point for the storage and elaboration of angiogenic regulators that are critical in both healing and “cancering” responses (Am J. Path. 2008 December; 173(6):1609-16). It is reasonable to conclude that many other critical elements important to cancering and healing responses would be stored in platelets, and released via similarly selective mechanisms. An assumption made in this disclosure is that successful wound healing is favored when the natural repertoire of factors needed at the site, is successfully delivered in a properly balanced manner. Conditions of inadequate wound healing, in which the patient's body cannot appropriately deliver these factors to the wound site naturally, are likely to maximally benefit if a therapeutic composition extracted and crafted from platelets, to match a natural optimal composition, generally recognized from research on that condition, integrated with specific diagnostic information the practitioner has on the patient's condition (in line with the healing trajectory concept), can be provided to the site by a practitioner.

In view of the above discussion, the product of the '963 patent may unnecessarily increase risk of cancer if applied in the incorrect contexts, in excessive dose, or with improper release kinetics, in comparison to more balanced mixtures produced from the same starting material. Clinicians skilled in the art would recognize that the product of the '963 patent applied in an infirm patient would require them to evaluate the degree to which the patient can elaborate their own subsequent, ideally balanced, antagonist response to compensate for those elements presumably lost in the dialysate, such as from their own platelets. A low platelet count should therefore be among those conditions considered to be a “red-flag,” suggestive of caution, in the use of products created with the '963 patent. Other relevant conditions can be envisioned by the person of ordinary skill in the art, where caution using the '963 product is advised.

Even experienced pathologists sometimes mistake normal wound healing response in a tissue for a cancering response. The distinctions can be very subtle and can impact a person's health, if improperly identified. A major motivation leading to the present invention comes from recognition that normal healing and the cancering response, as well as any chronic disease process, probably are strictly distinguishable through aspects of the balance of signaling events in the body, such as typified in the elements in platelets, their dynamics at wound or tumor sites, and anywhere in the body, simultaneously. The paradigm we prefer for moving toward making the correct distinction was used by Alan Turing to decrypt German U-boat communications during WWII, at first a seemingly un-interpretable mix of signals the British could intercept. The proteomics field at the present point in history seems to be following a paradigm comparable to the case in which the British decided that more sensitive receivers for the German signals would reveal the solution. Data exists in biomedicine, but little effective integration seems to be occurring. The birth of babies and the remarkable regenerative potential of the human body attest to the fact that a meaning exists in the mix of signals in our body, typified, but not limited to, platelet factors involved in development and healing responses, as well as cancering and chronic disease states. The instant disclosure seeks to begin to suggest the power of logical comparisons and rational integration of the signals to get to the deeply imbedded structure within the apparently noisy signals, that can underlie the difference between a desirable healing response and a “cancering,” or chronic disease state. The concept of a “ban” as a unit of information defined by Turing, that predates Claude Shannon's “bit,” seems to embody the best way forward in the cryptanalysis of human disease processes. This practitioner believes that the ban concept is more generalizable to weighting of evidence when it is not easily convertible into a mathematical form yet, such as is typical in the early characterization of biological phenomena, like angiogenesis.

If failed healing, cancer and chronic disease are opponents in a contest, then the presently disclosed inventions are the tools to find their “blind spots” (analogous to the location of the U-boats) and to enable some measure of mastery over them to a degree currently not possible using only the reductive, mechanistic scientific approach. This could amount to better, more rationally guided use of available medications and therapeutic strategies with platelet constituents

These inventions were informed by the notion that there may be a mistake inherent in the idea that any biological pathway or phenomena will ever be understood to the point that it can be controlled or mastered, in absolute terms. Our perspective has been informed primarily by pitfalls inherent in the idea of defining the process of angiogenesis in the body in terms of a set of signals, first and foremost in the literature being VEGF. Since evidence exists that pathogens can block or modulate angiogenic activity (Plos Pathog. 2009 May; 5(5):e1000420 Plos One 2011; 6(5):e20204) and are widely known for prowess in evolving new molecules in wound environments, it follows that any critical pathogen must direct its attention at the reading of signals used in our bodies to precipitate an angiogenic response with the intent to block or favorably modulate those signals, to it's own ends. In other words, a deterministic mechanism for regulating angiogenic activity would be met by pathogens evolving a repertoire of blockers, or regulating factors, that allow it to master this critical control point in the human body's defenses. This knowledge could be passed on to other pathogens, essentially leading to our selection out of the ecosystem in a manner akin to a monoculture.

These ideas have been informed by the idea that humans survive infections because our immune systems can elaborate a diverse repertoire of angiogenic factors based on the dynamics in the wound environment that are tailored to counter specific infectious threats present. The simple conclusion is that the means that our cells and organs use to communicate with one another must be “encrypted” in some manner, currently at the boundary of our understanding, for our survival.

David Agus describes the similar absurdity in the view that we will ever be able to have complete “knowledge” of cancer in his TED talk (A New Strategy in the war on Cancer): (available online Jan. 5, 2014).

Just as the Germans could keep shifting the Enigma rotor settings, and even create a new encryption keying system anytime, thwarting Allied attempts to understand or define in some absolute sense, any critical control aspect of the systems underlying our survival never can be fully defined, because if they could, it would represent a blindspot in the unfolding dynamic with other organisms. It would be akin to always playing a particular chess piece the same way. In such an example, when the opponent learns that fact, the piece then becomes useless in the game. Analogously, nature would have selected the particular biomolecule as metabolically costly deadweight. “Truth” gets evolved around in biological systems and, in the end the “truth wears off,” to quote Jonah Lehrer in his New Yorker article (available online Jan. 5, 2014).

The best Turing's methods could give the Allies was a window of time, before the German's learned that their codes were being broken and they could adjust the keying. Turing's system allowed Allies to become less and less incorrect about the meaning of the signals, but always playing a cat and mouse game when rotors and keying were adjusted, yet still a factor likely decisive to the Allied victory. The imperative for speed in the execution of logical comparisons that could be made became the essential factor necessitating designs leading into a modern computer. Turing's method has been considered to amount to a Bayesian inferential methodology.

These innovation have been informed by the idea that the Bayesian approach is naturally employed by engineers: they build a prototype, destroy it to find weaknesses, build a new one, destroy that to find more weakness, continuing in this pattern to continually improve the design. The same idea can be applied to hypothesis testing, to seek a truth in a complex dynamic system. The mode that a detective uses to solve a crime is analogous: take stories from witnesses, look for inconsistencies between the stories, ask more questions rationally guided by the inconsistencies, regroup and weight on new evidence, and seek more inconsistencies. Eventually, a useful image forms in these methods.

Thus, the processes herein disclosed, and later embodiments with significant refinements already envisioned, will likely have great diagnostic value, when applied to the extraction and further analysis of platelets, or any fluid or tissue dispersed in water, from patients suspected of having cancer. The diagnostic power of these processes will come from dual analysis of the fractions generated using this new invention, and refinements, in 1) holistic analytic methods such as the Matrigel plug assay and for diverse bioactivities (described, in limited, non-exhaustive terms below), that are known for roles in both “healing” activities, and cancering/chronic disease states, and, 2) in the types of proteomic analysis strategies being pioneered by Danny Hillis and David Agus, currently. This will yield a “logical process” tool that amounts to a kind of “microscope” or window into the abstract world of “Emergence phenomena” unfolding in the body in relation to factors contained in platelets, or other elements of critical signaling networks, as pertains to cancering and wound healing processes. This innovation represents a set of concepts Agus and Hillis appear to be missing in their efforts to date,

The 2011 review by Alan Nurden, “Platelets, Inflammation and Tissue Regeneration” (Thromb. Haemost. 2011 May; 105 Suppl 1:S13-33.) highlights the “duality” of regulatory factors in platelets across a variety of important bioactivities at wound sites (each generally seen to be distinct from the process of angiogenesis by other researchers), and each of these types of elements (agonist/antagonist pairs) can influence optimum healing outcomes. These elements also can take part in, and reflect, imbalances involved in “cancering,” a term traced to David Agus and Danny Hillis, to emphasize that cancer is a process, rather than a thing.

A major element of the theory behind this disclosure is that all the factors that bring about the angiogenic response also control the activation of the other systems Nurden broadly, though not exhaustively, details. These systems include, but are not limited to, the complement system, proteolytic enzymes (MMP/TIMPs), fibrinolysis, and coagulation. The list can also include inflammation, immune system functions, oxidative stress control, and probably even pain control. In short, everything occurring at the tissue and whole organism level regulated by diffusible signaling factors of any kind that is essential to outcomes in the balance between health and disease states following injury or stress.

Warren Weaver's distinction between “disorganized” and “organized” complexity (see Weaver's paper “Science and Complexity,” found on “philoscience” site online Jan. 5, 2014) provides a good basis for starting to understand these concepts. Basically, every element in the mix can contribute more than one function, and likely has a distinct role in each activity listed above. Data indicate that angiogenic response in vivo is the most critical phenomenon to measure, and is an effective proxy for all other activities. In other words, processes that result in platelet mixtures which maintain the optimal embodiment of natural angiogenic activity, (i.e. a theoretical limit these efforts are seeking, but may never practically achieve in absolute terms) required at a particular site of injury also preserve all of the other elements of activity listed in, but not limited to, the Nurden paper (Thromb. Haemost. 2011 May; 105 Suppl 1:S13-33.), optimized to bring about an “overall ideal healing response” at that particular site. Measuring an ideal angiogenic response from an isolate of platelets is akin to perceiving the stunning fruit qualities that great wine can have, and that signifies the winemaker did a good job; a product of eons of refinement in that art. The presence of stunning fruit on the pallet likely means other natural elements and aspects of the grape that can favor promotion of health and well-being have been collected and preserved as well, in a better manner than the wine made from the same grapes, but lacking the stunning fruit, resulting from the work of an inferior winemaker. A corollary of this concept in diet is to eat real, whole, fresh, natural, minimally processed foods, in the forms your ancestors consumed. These foods also contain a similar embodiment of nature's “emergent” wisdom found to be essential for human health, as detailed in research on optimum human diets, across cultures and history going back thousands of years. Just as the fruit of the barley plant is transformed into a product of human ingenuity once essential to human survival, through the guided activation of latent enzymes systems, so too are platelets a fruit of the human body, containing enzymes involved in converting nascent elements into an essential “emergent” form guided by stresses and dynamics at wound sites, to enable survival after injury.

The idea that “cancering” and healing responses can be broadly conceived as the flip-side of a single coin was probably first described in depth by Dvorak (NEJM 1986 Dec. 25; 315(26):1650-9). Integration of these concepts has been critical in the conceptualization of this innovation. The same issues of “organized complexity,” and “emergence” are critical elements to be recognized in regenerative medicine and this has informed these new approaches.

The agonist and antagonist factors of each activity (not just angiogenesis), that can be found in platelets, or biological materials generally, have known and distinct, molecular weights, which serve as the basis for the separations described below, using dialysis membranes of defined molecular weight cutoff (MWCOs).

### BRIEF SUMMARY OF THE INVENTION

We propose five new processes, and/or refinements, of existing art:

1) Take the excluded material from dialysis (dialysate) generated as described in the '963 patent and allow it's reintegration, in defined portions, directly into the product of that invention. Performing this procedure with a variety of MWCO membranes, for example, other than 6-8 kD, provides a way to determine the relative contributions of specific low MW elements, to the infiltration of acidity into the extract as a controller of the resulting bioactivity of the product due to effects of acidity on conversion of latent elements to active forms.

2) From a first patent, dialysis was usually done to exhaustion, meaning the dialysis bags containing about 50 ml of extract went through multiple (2-4) 1 Liter exchanges. If only a single exchange is done and the exchange volume is reduced, this is a way of reducing the concentration of the lower weight elements left in the product, without exhaustively depleting them, essentially achieving the same end, as in 1) above, but without adding back anything. For example, if a single exchange is done in a 200 ml bath (for a 50 ml extract in the dialysis bag) then the final concentration of the low molecular weight elements would be reduced 5-fold. Thus, the exchange bath volume can be seen as a point of control for the amount of the low molecular weight elements left in the product, i.e. in the bag) after the process.

3) A “two-membrane process” is proposed for targeted depletion (exclusion) over a range of molecular weights from the extract. This process is similar to the use of filters for signal processing in the design of electronic circuitry, with “high” and “low” pass elements properly utilized. In the present case, the pure, unmodified “signal” is the whole lysate after removal of insoluble elements by centrifuging, but prior to dialysis. If, for example, we wish to create a product that selectively excludes product between molecular weight A (on the low side) and molecular weight B (on the high side), we obtain dialysis membranes with MWCO A and MWCO B. We perform the dialysis of the lysis supernatant in bag A and retain the dialysate (retained dialysate) to the level of exhaustion desired, as per 2), above. Next, we transfer the retentate from bag A to bag B and re-dialyze, also to desired exhaustion, retaining the contents of the bag and generating an “excluded dialysate,” as well. The contents of bag B is then combined with the portion that was retained as the flow out of membrane A (retained dialysate). In the case where the molecular weight of A equals B or A is greater than B, the process approaches the same theoretical compositional outcome as in 1), above, when the entire dialysate is added back to the product.

4) If the “two membrane” process is applied iteratively, elements over an unlimited number of defined zones of molecular weight can be depleted and collected into excluded dialysates. A method for very precise control over the composition can be achieved if the membrane MW cutoffs can be very precisely defined. For example, retained, or excluded dialysates can be added into un-depleted portions of an extract, to effectively increase the concentration of those elements in some products. A bone-forming product could be envisioned by doing this selectively for BMP and TGFbeta-superfamily factors desirable for osteogenic activity. Mixtures depleted in certain elements, and with augmented concentrations of other elements can be conceived to perform as “idealized” mixtures that would be elaborated at a wound site, but for a particular disease state associated with the failed healing state that the patient is suffering.

5) A one-membrane process can allow another means of removing elements over a range of MW, or based on biophysical affinity for the membrane, in the finished product, but without the need for two membranes. This embodiment takes advantage of the fact that a portion of factors enriched in the MW range of the membrane (6-8 kd for experiments presented here), could be removed because of their retention within the matrix of the bag, upon it's removal from the finished product. This is based on the idea that the space inside the dialysis membrane will have become relatively enriched in a certain fraction of factors within the MWCO range, and also based on these factor's particular biophysical affinity's for the membrane material, with those elements of MW above the MWCO relatively excluded, and retained within the membrane bag, and those of far lesser MW, allowed to diffuse freely across to achieve an equilibrium on both sides, and thus relatively depleted overall, relative to the fraction of factors inside the bag (MW>MWCO) and those inside the membrane. Thus, the choice of MWCO of the membrane, and membrane material, holds a means to deplete a portion of elements over a chosen MW range, and/or based on biophysical affinities that lead to a relative increase, or decrease in concentration of various elements within the membrane. The relative volume ratio of the membrane, to the total contained, and external volumes would have to be considered in gauging the overall percent depletion. Subsequent rinsing to various degrees, or digestion of the membrane, to capture, or determine contents removed from the platelet extracts remaining, would be important in making such a separation approach effective. Collection of a product from the membrane itself after removal from the contained and external volumes based on these principles can provide a reciprocal method for relative purification of elements from the overall extract, presuming development of processes to elute them from the membrane material subsequently. This can simply amount to placing the membrane in a bath of 10 mM HCl for them to dissolve out. Other dissolving solutions can be envisioned, to allow optimal elution based on the particular affinity between desired elements and the membrane material properties include the MWCO. The thickness of the membrane and material chosen can be envisioned as critical parameters in this implementation, depending on the desired separations.

The power of these new processes can be elucidated by study of the products in various assay systems for tissue regeneration and cancering processes.

### BRIEF DESCRIPTION OF THE SEVERAL VIEWS OF THE DRAWINGS

FIG. 1, on pg 51, is a graph showing the direct comparison of material from 3 paired platelet units made with the '963 patent process and the New Processes applied in a graded fashion, and evaluated in terms of an MTT assay for cellular metabolic activity of human dermal fibroblasts cultured for 7 days in media supplemented to 4% with the products. The MTT reading can be considered an index of overall cellular viability and metabolic health in this setting.

This drawing summarizes the cellular metabolic activity evidence showing superiority of the new processes over the '963 patent process, that also mirror morphological findings on the same materials. Shown in the drawing on the left, labeled as “old process” are the average reading and standard deviation of the triplicate MTT assay values from groups 7, 8, 9, which were treated with '963 patent process material. On the right, above the label “New Process,” are the average and standard deviation of the triplicate MTT assay values for groups 10, 11, and 12, which were treated with products of the New Processes. These groups could be considered to represent the best direct comparison possible between the '963 patent and New processes, by those skilled in the art. These results are mirrored by ranked morphologic evidence presented below, that also suggest that the New Process material does a better job than '963 process material in terms of supporting maintenance of advantageous cellular phenotypes, enhanced secretion of critical matrix elements, and production of more highly organized fibrillar forms, that are generally considered by those skilled in the art, desirable and valuable in the restoration of tissues including, but not limited to, dermal, tendon and ligament organs following wounding.

### DETAILED DESCRIPTION OF THE INVENTION

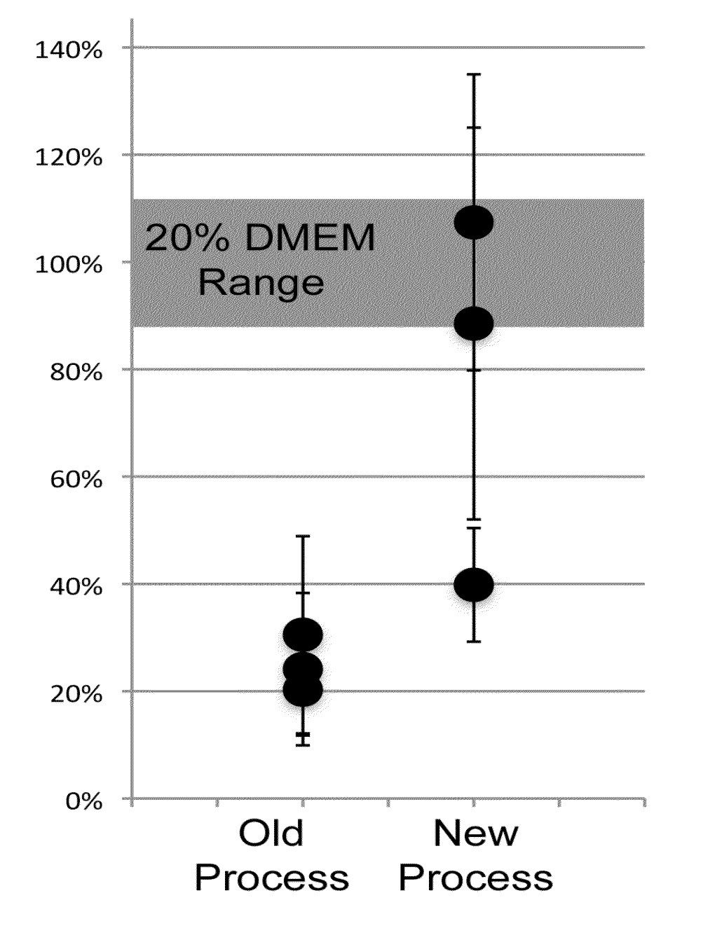

Materials created by these new processes applied to human dermal fibroblasts in culture reveal improvements over the earlier '963 patent product in terms of quantitative MTT assay readings, reflecting improved viability and overall health status of the cells, shown in FIG. 1, pg 51, which shows a summary of the most relevant evidence from Data Table 2, on pg 37. Matched, ranked visual evaluations of key morphologic findings corroborative of the MTT evidence are presented in Data Table 3, on pg 42. An additional piece of morphologic evidence suggestive of the superiority of the new processes was the presence of what appeared to be a fine precipitate on the culture surfaces of groups 1-5 and the serum groups. One interpretation of this finding may be of unregulated oxidative stress in the cell culture media environment leading to oxidation of proteins, their denaturation and subsequent precipitation from the media. The lack of these finding in the groups treated with higher doses of the '963 product (7, 8, 9) as well as the groups treated with the New Process material (10, 11, 12, 13, 14, 15), which also showed better cellular viability, and other morphologic evidence of robust phenotype expression, would be consistent with an interpretation of oxidative stress out of balance in these lower numbered groups. The finding of these precipitates in the serum treatment as well, suggests the importance of the new processes and products in facilitating progress beyond the standard in these areas of art.

### Example 1

### Restoration of the Imbalance

First, a cream or ointment made from the product described in the '963 patent, plus all the excluded factors in the dialysate can be added back in exactly the same ratio present in the beginning platelets, likely will be safer and more efficacious for topical skin and ocular applications in healthy patients. This “natural” version of the product may have lower angiogenic activity/dose in the Matrigel plug assay, but other parameters of healing efficacy and safety are likely to be more optimal, since the natural balance of other aspects of healing activity are restored, which were altered in the creation of the '963 patent product that possibly hinged on an imbalance created by the process, resulting in augmented angiogenic activity. These “natural” compositions can be tested in cell culture and in other assays for the various activities discussed to a limited extent in the Nurden paper (Thromb. Haemost. 2011 May; 105 Suppl 1:S13-33.). This is identical to the formulation of botanicals into creams/ointments or unguents for skin wound healing. Humans share a common ancestor with plants going back about 1 billion years, which may be a reason that botanicals can have a positive healing impact on humans, generally.

This invention also can have value in eye-drop formulations for optimizing healing, with less infectious risk and reduced scarring following ocular surgery and other ocular indications, such as dry-eye syndromes.

This invention can be useful for treatment of diabetic ulcers and other chronic skin wounds. Treatment of laser-resurfacing wounds created for cosmetic reasons also would be improved.

This invention can yield an ideal quality controlled version of platelet-rich plasma. Platelet-rich plasma is widely used in sports medicine injections and orthopedic surgery. As injectables, these embodiments have the advantage of providing a high concentration of growth factors in water, which translates to the use of narrower gauge needles and smaller, more precise volumes injected. This could be particularly important for difficult cases such as Achilles tendon injuries. Combination with a bone graft substitute could result in a bone-forming product. Dose in a small amount of rhBMP-2 and a better product than the high dose embodiment of Infuse for spinal fusion would likely result and could be developed with a rodent osteogenesis assay.

The product can be useful for dental perioplastic procedures to augment the gingival or bony tissue around teeth for cosmetic purposes and to reduce the risk of tooth loss.

These embodiments can be useful in an austere care setting, where a patient is able to return home with the product and instructions for its application, or the practitioner applies or injects the product in the office.

This invention can be useful for treatment of neurotrauma such as spinal cord injuries or stroke traumatic brain injury (TBI), or any of the neuroprotection/neuroregenerative applications pioneered by the compound called Cerebrolysin (Drugs Today (Barc). 2012 April; 48 Suppl A:43-61) in humans and animals.

These embodiments are likely to reveal other broad fields of use, such as cardiovascular, artificial skin, and neuroregeneration/neuroprotection. A product in development by Theregen, ANGINERA®, is a cellularized patch that can be applied to the ischemic heart. The extracts formed by processes of the present invention, incorporated into a matrix to form the patch, likely would be safer, easier to quality control for efficacy and less expensive to make. The same concepts should extend and inform creation of advanced embodiments of an “artificial skin” product along the lines of TRANSCYTE®, DERMAGRAFT®, or APLIGRAF®.

For neuroregeneration/neuroprotection, similar patch-type constructs in which the extract is combined into some matrix material to be applied to the brain, over a region damaged by stroke or TBI, or into the spinal space close to a spinal cord injury, for controlled release of the factors, could provide a useful follow-on concept.

Another embodiment could be combination of the product with cells that are difficult to engraft to the human body for therapeutic purposes. These could include pancreatic islets, cardiomyocytes, neural stem cells, any cell-based therapy or, specifically, adipocytes that might be injected to augment the fat pads on the feet. Attempts to place any type of cell, stem or otherwise into the human body for therapeutic purposes, generally are challenged to network the cells into the body's systems, connection to the vasculature being just one of the major aspects this invention may enable when properly used.

Combination with a collagen membrane, human tissue, or other matrix substitute device to heal, or create a seal in a tissue defect, such as anal fistulas seen in general surgical practice could be another embodiment.

### Example 2

### More Advanced Care Settings, Rational, Limited Restoration of the Imbalance

If the ratio of the excluded portion added back into the product is rationally guided by the stage and imbalance, or degree of impairment of wound healing which can be identified by a practitioner, in line with the “wound healing trajectory” concept known to those skilled in the art, then the invention can give a product with enhanced healing value, able to influence restoration of balance leading to a better healing outcome. Applying a mixture to create a desired initial response, such as shown in the '963 patent for wounds lacking sufficient pro-angiogenic stimulus (as one example), or any more carefully crafted mixture using the processes listed above (that can give enhancement or inhibition of other desired responses), one then selectively adds in, over the proper function of time, as judged necessary by the practitioner, at the wound, the antagonist mixtures from the excluded portions. For wounds lacking enough angiogenic stimulus, considered as a simple “first-order” example, this amounts to adding in the excluded dialysate elements presumed to include antiangiogenic factors that can antagonize the agonist response to the product of the '963 patent product, in line with the manner presumed to naturally occur in healthy wound healing, by the mechanisms described for release of different alpha granule populations by selective receptor activation on the platelet surface in the wound microenvironment (Blood 2008 Feb. 1; 111(3):1227-33). The concept is generalizable to all the agonist/antagonist signaling relationships that control critical activities in healing and cancering responses, which can underlie any particular impairment in healing, related to signals regulating the essential processes in platelets, and other tissues.

This methodology is analogous to that employed in the guidance system for piloting an airplane to a safe landing. Ailerons, elevators, and a rudder are the primary control surfaces in aircraft, but secondary surfaces can be included, and there is no limit to the number of different control surface modifications that can be built into an aircraft.

Consider the example of a dragonfly. Every aspect of the emergence of the organism, each “control surface,” a concept of infinite scale, comes into play over evolutionary time. Consider that a plane navigates within a “space” called the atmosphere, while the wounded, chronically ill or “cancering” body is navigating in another, at this time in history, more obscure “space” toward either a safe landing (normal healing) or potentially a crash, in the form of abnormal healing, i.e. cancering or chronic disease state. For the purpose of this disclosure it is presumed that the healthy body manifests the naturally optimal “control system” we are seeking to describe and uncover, to heal wounds and survive infections, in most cases without the need of a clinician. This same system is out of balance in cancer and chronic disease states. The system is likely unique between individuals, to some degree, and shaped by many forces in the individual's environment, and personal history. We are at an early stage in designing these controls for clinical application, but nature can act as a guide to help us become more and more correct.

From the concluding paragraph of Nurden's paper: “Yet, a major enigma remains to be resolved. Platelets have now been shown to store and release such a wide range of biologically active proteins that the question as to why such a wide diversity of compounds is required needs to be asked. Not only this, time and again in this review I have shown how platelets stock and release activators and inhibitors of each biological pathway; angiogenesis, coagulation and fibrinolysis being just three examples. Why should this be? Perhaps local conditions determine which wins, if so the regulation of the non-haemostatic roles of the platelet is indeed finely tuned.” (Nurden et al., Thromb. Haemost. 2011 May; 105 Suppl 1:S13-33.)

Each agonist/antagonist pair of signals in an organism can be viewed as conceptually identical to the potentially unlimited number of control surfaces on an aircraft, able to influence navigation of the organisms trajectory to a “state of health,” following injury or other stress, but also having critical roles during development. The question for the design of mixtures from platelets for therapeutic purposes, or the uncovering of the essential signaling imbalances at the core of a person's disease condition becomes one of determining when a given “agonist/antagonist” imbalance (like a control surface on a plane) is in need of adjustment, and where and at what time in the cycles unfolding in the organism, that balance needs to be restored, and in what direction, and to what degree. We believe Turing's concept of the “ban” and “banburismus”, in a more broadly defined sense, can be applied in an iterative manner for any given patient to determine the particular setting of these control elements, e.g. “elevator up” or “elevator down,” agonist vs. antagonist, necessary to favor a trajectory toward positive healing outcome, in a given setting, as opposed to cancering or chronic disease. The “ban” has to be applied to the weighting of potentially vast quantities of data, but it seems tractable to work on using the now more developed concepts of sequential analysis, known to those skilled in the art, to make headway in the decoding of a disease, in relatively short order, for an individual person.

From the Wikipedia page for “Ban_(information),” (available online Jan. 5, 2014):

Jack Good argued that the sequential summation of decibans to build up a measure of the weight of evidence in favour of a hypothesis, is essentially Bayesian inference. [1] Donald A. Gillies, however, argued the ban is, in effect, the same as Karl Popper's measure of the severity of a test. [2].

From the Wikipedia page on Alan Turing (available online Jan. 5, 2014)

Jack Good opined, “Turing's most important contribution, I think, was of part of the design of the bombe, the cryptanalytic machine. He had the idea that you could use, in effect, a theorem in logic which sounds to the untrained ear rather absurd; namely that from a contradiction, you can deduce everything.” Id. (available online Jan. 5, 2014)

The essence of what is revealed here stems from the fact that a simple dialysis membrane can be used to create a separation, based on a defined Stokes radius, from a soup of biomolecules in water to reveal, and allow to be collected in separation, an imbalance (a sort of contradiction) among agonist/antagonist symmetries naturally present in the organism, which are critical to the unfolding dynamic of either healing responses vs. “cancering” or chronic disease processes.

The hypothesis at issue around which a “weight of evidence built up to test its veracity” (ban) in the sequential analysis regards whether a particular component is contributing to a “healing” response or a cancering/chronic disease response. The component can be a single signal discussed in the literature, gradations in concentrations of that signal (with reference to the Morphogen concept), or an isolate of potentially many signals in a mixture. In all cases, these signals must be understood within a context, that the holistic “big-picture” side of medical practice, which at this time is frequently lacking in standard medical practice, must provide in the techniques conceived.

For example, the literature on disease processes such as pulmonary hypertension, or preeclampsia, are rich with information amounting to the signals in the body that pop up repeatedly in studies of these frequently fatal conditions. With this information and a practitioner's overall, holistically informed, clinical understanding of the patient's condition coupled to the innovations disclosed here as modes for the analysis of platelets, fluids and tissues collected from the patient for setting of the “ban” during the course of sequential analysis, it may be possible to decode the condition to reveal better information and guide more rational therapy of these diseases. With time, understanding of ways to deploy the entire approach to potentially more complex, pleiotropic disease states like cancer can be envisioned. A simple counter-top device for a physician's office or hospital able to do the essential membrane separations, with settings (molecular weight cutoffs) defined by the practitioner, can be envisioned. Physicians could become Banburists.

Just as “elevator up” at the wrong time in airplane flight could undermine stability, while “elevator down” could favor safer flight, so the practitioner should be able to determine, at each point in time and site in the body, which of the agonist/antagonist pairs needs to be shifted, and in which direction and to precisely what degree.

Dosing rhPDGF and other growth factors such as rhBMP-2 at grossly high levels relative to the physiologic norm is analogous to building and launching a rocket with a powerful booster, but with no guidance and control system for landing it safely. Analogously, the present invention gives insight into how to build the needed guidance systems for optimizing wound healing in clinical settings. The product of this embodiment could initially take the form of different tubes of cream/ointment/biomaterial made by a compounding pharmacist containing the distinct products, applied at different times, to severe burns, as one example. However, the concept is generalizable to any imbalance in the body that has led to a disease condition and can be deployed to restore the balance, once a practitioner knows the nature of the imbalance. With iterative application, an unlimited number of imbalances can be addressed rationally with the innovation.

The invention also includes incorporating the different portions into lyposomes, or some other material with distinct release kinetics for each portion, within the whole cream, so that the release profiles match that desired by the practitioner after assessment of the wound, to determine an ideal trajectory to a good outcome. This aspect employs the type of imbalance characteristic of products from the '963 patent, as an advantage in creating balanced healing.

The imbalance of angiogenic activity characteristic of the '963 patent separation process is only one specific type of imbalance a MWCO-based separation can expose in any mix of biological molecules in water. If the MWCO is chosen appropriately, any particular “critical bioactivity” regulated by signals in platelets or other biological fluids or tissues, can become imbalanced by the separation, since the MW of agonists and antagonists for that bioactivity are generally different. Thus, on each side after separation, there might be more agonist factors, while on the other, more antagonist factors for that aspect. The critical factor in exposing these imbalances is the bioassay for a particular activity to establish when the MWCO has been found that exposes the imbalance. Thus, the terms “agonist” and “antagonist” are intentionally kept as generic concepts, not limited to angiogenic activity, to highlight the fact that this invention, based on simple choices of MWCO of the membranes, can create separations that allow for any of the key activities detailed above to be targeted. The key to exploiting these processes hinges on the quality of the bioassays for the critical “cancering” or healing activities to confirm that the separation has led to the exposure of the imbalance. Once the imbalance is exposed, it can be used to selectively restore balance in clinical circumstances as identified by the practitioner for wounds characterized by that type of imbalance.

Prior work in this field has been heavily informed by research in cancer biology focused on the study of “angiogenesis” from the perspective of a search for individual growth factors that can either stimulate, or inhibit the growth of new blood vessels, beginning with the work of Judah Folkman (J. Pediatr. Surg. 2007 January; 42(1):1-11). From this perspective, angiogenesis is viewed as an abnormal process resulting from chaotic or disorganized elaboration of factors, distinct from more normal modes of generating a response needed during development, or following traumatic injury leading to a proper healing response. Single factor blockers of angiogenesis, widely known to those skilled in the art, for cancer and chronic diseases, and the subsequent effort to translate single factor angiogenic agonists for therapeutic application (“therapeutic angiogenesis”), have generally shown limited utility, suggesting that our understanding of the phenomenon, with respect to normal healing responses, has been lacking. These approaches have been informed by the idea that the current understanding of the word “angiogenesis,” has limited the advancement of tissue regeneration strategies, and that a deepening of our view to blood vessel biology from a more holistic perspective is needed. A better term, which provides some expansion of the concept of “angiogenesis” into the area of definition we seek, is “vascular normalization,” which is becoming recognized for it's importance in cancer therapy (Front. Onc. 2013 Aug. 15; 3:211; Vasc. Cell 2013 Dec. 6; 5(1):20). Seen from the near polar opposite perspective of tissue regeneration, this term can essentially equate to “re-establishment of proper blood vessel function and regulation,” which is what is needed more generally at wound sites, which can be seen as including, but is not properly limited to, new blood vessel sprouting and growth behaviors, which are most easily quantifiable, but not identical with the whole picture. This phenomena could also include, for example, cases of injured but basically intact vasculature that has become leaky or dysfunctional in some manner that needs to undergo a re-stabilization of the basement membrane encapsulating the vessels to support return to optimal phenotype of mesenchymal and endothelial cells composing the structure. Thus, one skilled in the art could assert that optimal wound healing involves a natural mode of “normalizing the vasculature” following injury, and that this is a property which manifests, in part, from combined behaviors of natural mixtures in tissue matrices, and platelets present at the site.

The ability of natural isolates of bone-derived factors to favor formation of more “normal,” and less leaky vessels than single factors in vivo was shown in a 2005 paper (Angiogenesis. 2005; 8(4):327-38). The evidence shown here from cell culture comparison with the '963 process supports an idea that these materials can favor “normalization” through beneficial influences on fibroblast phenotype, reflected in part in improved elaboration and remodeling of extracellular matrix elements, which can be observed in the form of aligned collagen bundles, and basement membrane elements critical to the differentiation of vascular structures. One skilled in the art could assert that a whole tissue (in this case a dermal organ) must be seen as inseparable from vascular elements, for advancement of these arts.

Auerbach et al. also emphasized the need for a more holistic view to the assessment of angiogenesis (Clinical Chemistry January 2003 vol. 49 no. 1 32-40). They note that the Matrigel tube forming assay, which is most commonly used to evaluate tube forming responses of endothelial cells, can also reveal the property of fibroblasts to form tubes on Matrigel, which those skilled in the art would see as an “angiogenic” property of these cells, i.e. showing their contribution to an overall process leading “vascular normalization.” “Vascular mimicry” is a term, recognized in the art that can describe such behaviors across multiple cell types. In vivo work with bone-derived factors added to Matrigel plugs suggests that both fibroblast and endothelial cells are forming tubes in the plugs in the process leading to formation of the mature capillaries we observe (Angiogenesis, 2005; 8(4):327-38. Epub 2006 Jan. 7). This is in line with histological analysis of plugs loaded with growth factors, other than the natural platelet extracts described here, by Anghelina et al. showing intimate cooperation between cells of different lineages, some endothelial, others likely fibroblast lineage, deriving from blood borne cells of monocyte/macrophage lineage, in the formation of capillaries and tubular structures (Am J Pathol. 2006 February; 168(2): 529-54; Vasc. Cell 2011 Feb. 2; 3(1):4). A particularly notable finding was that collagen bundles were associated with cells of an apparently tubular character, perhaps fibroblasts migrating along these bundles, revealing a patterned response, that facilitated later migration of cells along them and tubular morphogenesis along the path. This suggests that fibroblasts play a critical role leading into initial luminal forms, through secretion of organized extracellular matrix in the form of collagen bands. They also propose the inadequacy of models that describe neovascularization responses in terms of behaviors of endothelium or endothelial precursors alone. Thus, the expression of normal, or optimal phenotypic characteristics of fibroblast cells, responding to local growth factor mixtures, is a critical element of “angiogenic” response, even as defined in both cell culture and in vivo settings. Whether the terms “angiogenic activity” or “vascular normalizing activity” or “regenerative enhancement activity,” are most appropriate to the findings reported here, would seem a valid subject for debate, in this evolving field. Since the Matrigel plug assay provides a view focused on new capillary growth, in line with the standard for “angiogenic activity” (newly formed functional capillaries defined by differentiated endothelial cells), as do other current quantitative in vivo angiogenesis assays with primary focus on the endothelial cell, this type of analysis would not give results more useful than those reported on human dermal fibroblasts in culture to reveal more subtle aspect of tissue regenerative activity.

These inventions have been informed by the idea that the Matrigel plug assay would likely reveal a lower (or less enhanced) angiogenic response of the product, compared to the product of the '963 patent, because these new processes can be seen as “balancing” the product of the '963 patent, and therefore the plug assay is less suited to discriminate the influence of process changes than could be found in direct application to human cells normally involved in human tissue morphogenesis. Those skilled in the art would conclude that these results have a broader relevance to tissue regeneration, and vascular biology, than described by a traditional view to “angiogenesis,” that is restricted to endothelial cell behaviors and the sprouting growth of new capillaries in vivo. An attempt to seek a more broad and nuanced view of the field, and the term, “angiogenesis” is suggested.

### Example 3

### Application of '963 Process Material in Human Cell Culture

Two lots (from two separate platelet units) of '963 process material were made and compared to material made using lysis in PBS supplemented with a standard protease inhibitor cocktail mixture, but with no subsequent dialysis step. Primary human fetal skeletal muscles cells were placed in culture with high glucose DMEM supplemented only with platelet extracts, or FBS under conditions that led to confluence, or near confluence in platings with 10% or 20% FBS, after 5 days. Platelet extract supplemented cultures, which had not reached confluence, were judged on gross morphological criteria, in terms of overall cellularity (approximate coverage of the plate with cells) and the presence, or absence of accumulated debris, or apparent balled up cells, likely indicative of effects due to high growth factor activity applied in the media. The platelet extracts were dosed in the range of 1.0, 1.5 and 2.0 mg/ml (ie approx 18-30%, since ave extract protein conc.=4-7 mg/ml). Accumulated debris of evidence of balled up cells tended to be higher in the higher dose treatments. Cells treated with platelet extracts showed spindle, or branching morphology typical of mesenchymal cells in surface culture. Judged in terms of higher cellularity, and lower appearance of debris in the images, the PBS+protease inhibitor cocktail treated extracts tended to perform better at equivalent dose, or on par for a slight increase in dose of the '963 process material. The observed differences can be considered for the fact that a low molecular weight portion of factors would be lost in the '963 process, which should be present in the PBS+PI treatment, and that the extraction in neutral water alone, and therefore lacking buffer stabilization, followed by gentle influx of acidification across a dialysis membrane, in the '963 process could provide for the inactivation of proteases, which the addition of inhibitors in the PBS+PI treatment handles.

The inventions revealed here were, in part, informed by the idea that there may be ways to improve performance in primary human cell culture of materials made by water extraction followed by dialysis, and without the need for added protease inhibitor cocktails, or use of physiologic buffers such as PBS for extraction. These inventions were also informed, in part, by awareness that the mashing of malt requires only the addition of water at the correct temperature, and volume, and that these conditions lead to mobilization of natural buffering elements in the malt to maintain a proper pH, and that careful maintenance of the mash under these conditions, over defined periods of time, are all that is required to achieve desirable transformations.

### Example 4

### Application of '963 Process Material the Matrigel Plug Assay in Mice

A 2011 publication details in vivo and cell culture activities of the '963 process material (Vasc. Cell 011 Feb. 2; 3(1):4). Notable findings were the dose-dependent angiogenic activity of extracts in Matrigel plugs implanted in mice, as well as the enhancement of activities with addition of adenine nucleotides, the type of small MW elements that could be lost from the final product in the '963 patent process.

### Example 5

### New Process Evidence: A Direct Comparison of Products from New Processes

To '963 Patent Products in Human Cell Culture.

Methods: Three units of expired platelets (A, B, C) were obtained by courier mid-afternoon of the day following midnight expiration. These were aliquoted within a laminar flow hood into 50 ml conicals and pelleted using a benchtop centrifuge at low speeds (2000, 2500, 2500 rpm) for 20, 25, and 20 minutes, respectively, to pellet the platelets. The serum was carefully pipetted off each pellet with care to avoid disruption. A total of approx 90 mls of sterile water was distributed equally on top of the pellets, for each unit. (typical examples: if 6 tubes were used, each tube received 15 mls; if 8 tubes, each might receive 10-12 mls). The batches were placed in a benchtop bath sonicator for 60, 60, or 40 secs, respectively. Following a second centrifugation at 3000 rpm, for 25 minutes, the supernatants were collected and re-pooled. At this point material from each unit was divided and run either through the old process, by putting 40 ml of the isolate in a 6-8 kD dialysis bag, and carrying through two 1 L exchanges of 10 mM HCl over a 2-day period, or taken through the new processes. Each involved overnight refrigeration with gentle agitation or stirring. For the new processes an equal 40 ml of the isolate was again placed into a 6-8 kD dialysis membrane and dialyzed against 300, 350, or 400 ml of 10 mM HCl. The following day the material inside the bag, and in the larger dialysis solution, were allowed to gradually reintegrate by opening one of the clips on the bag and placing back in the container. The opened bags were removed after a period of time, likely in the range of an hour to a few hours later, allowing the remaining material in the bag to flow in to the larger volume, and the small portion of material in the liquid phase hydrating the membranes to be removed, along with the membranes. Testing for pH using test strips showed that materials resulting from the old or new processes had a pH in the 2-3 range, which is typical for the material resulting from the old process. A portion of the old process material was diluted 10× with 10 mM HCl in preparation for comparison with new process material in cell culture. Materials were sterile filtered through standard low protein binding membranes used in cell culture techniques, and maintained stability (i.e. showed no overt signs of precipitation) in a refrigerator over the one week test period. Base media was DMEM with 4.5 gm/L glu,+1-glutamine, and 110 mg/ml sodium pyruvate.

Media containing platelet lysates maintained the same color after addition of all extract variants, up to the point of media changes, indicating minimal effect on pH of the media bathing cells across all the processes used. Slight color changes were noted in the serum groups at feeding, suggesting some change in pH in these treatments resulting from metabolic processes of the cells. Normal, human dermal fibroblasts were plated at 60,000/well in a 96 well plate in a standard media volume of 300 microliter. After one day, base media was changed to include the test media. Feeding regimen was Mon, Wed, and Fri. Cells were maintained in standard mammalian cell culture incubation conditions. An MTT assay (Cayman Chemical Co, Ann Arbor, Mich. item 10009365) was run at 7 days. Visual assessment at this time was consistent with cells having maintained approximately the same number as plated across all groups.

Note: In this setting of altered growth factor support, the MTT assay can be interpreted to provide a robust “measure of viable cells (Berridge et al. 2005, p 141.)” The author goes on to say: “changes in growth conditions including growth factor, hormone and serum supplementation, and addition of cytotoxic and cytostatic drugs will alter the metabolic signal in a way that gives useful information about the effect of the particular compound or extract.” (Berridge et al. pg 141, Biotechnol Annu Rev. 2005; 11:127-52). The author also raised concern over the use of Tetrazolium salt assays for measurement of cellular proliferation and it's inhibition, given the diversity of sites of their reduction, and challenges with interpretation of the findings (pg 141). It should be noted that cells can use energy to proliferate or differentiate to variable degrees, and attempts to reduce one aspect of a cell-based assay system to biochemical reduction on a single molecule, may be problematic, without corroborative evidence. It should also be considered that influences on proliferative cells may not be an ideal model of safe wound healing, in which regulated proliferation, with proper tissue differentiation may be more desirable. Defining activity in terms of influences on proliferating cells, may lead to identifying, or defining “best” processes as those which produce materials with excess proliferative activity, and consequent cancer risks. Thus, we performed the MTT assay on fibroblast cells displaying little proliferative activity and also evaluated morphological evidence of dermal organ developmental processes.

Note on comparison with animal serum: It should be considered that the standard for many years has been animal serum-based media. This means the terms “ideal culture conditions”, and “ideal isolation conditions” have been defined largely around what works well with animal serum. Thus, various supplements in DMEM, as well as the pyruvate, glucose, and 1-glutamine, may actually not be to the benefit of a new, arguably more natural, media source such as from human platelets (ie. less need may exist to add other factors that have been necessary for work with animal serum.). This may be reflected in the lower variation between the three MTT readings for the control (20% DMEM, see below), relative to the variations for the platelet extract batches. A 2% addition of the old process material to media was chosen for reference to earlier work with these extracts in culture. Our observance of slight color changes in the serum groups, with no noticeable color changes in the platelet extract groups, could be reflective excess metabolic stress resulting from the serum treatment, relative to platelet extract treatment, potentially revealing a subtle benefit of the use of these, arguably more natural materials, relative to standard supplements, for primary human cells. Since the new process involves dilution due to incorporation of the solution outside the bag, we controlled for this difference by diluting the old process material to a comparable degree (10×) using vehicle (10 mM HCl). The mass of protein lost in the '963 dialysis process is worth accounting for, since it is reintegrated in the new process. A reported protein mass of 2.6+/−0.6 pg/plt was found in the literature (Blood. 1983 October; 62(4):924-7), and our typical finding of 4-7 (ave. 5.5) mg/ml resulting from a single unit extracted by the '963 patent process in a typical 70-90 mls, gives an average yield of (5.5×80=) 440 mg/unit. At 3×10 to the 11^{th} plts/unit this means a total protein content averaging 780 mg/unit. Since the pellet after the second, higher speed spin, typically appears more than half the size of the original pellet, prior to extraction, it seems reasonable that a large fraction (on order of >85-90%) of the total extracted protein is accounted for in what remains in the dialysis bag.

DATA TABLE 1
MTT Assay - Group designations of platelet
extract supplemented media were as follows:

```
“Un-numbered” control group - 20% animal serum supplemented DMEM
  1) old (‘963 patent) process, 1x, donor unit A, 2% in DMEM
  2) old (‘963 patent) process, 1x, donor unit B, 2% in DMEM
  3) old (‘963 patent) process, 1x, donor unit C, 2% in DMEM
  4) old (‘963 patent) process, x/10, donor unit A, 4% in DMEM
  5) old (‘963 patent) process, x/10, donor unit B, 4% in DMEM
  6) old (‘963 patent) process, x/10, donor unit C, 4% in DMEM
  7) old (‘963 patent) process, x/10, donor unit A, 8% in DMEM
  8) old (‘963 patent) process, x/10, donor unit B, 8% in DMEM
  9) old (‘963 patent) process, x/10, donor unit C, 8% in DMEM
10) new process, 1x, donor unit A, 4% in DMEM (dialysis vol = 300 ml)
11) new process, 1x, donor unit B, 4% in DMEM (dialysis vol = 350 ml)
12) new process, 1x, donor unit C, 4% in DMEM (dialysis vol = 400 ml)
13) new process, 1x, donor unit A, 8% in DMEM (dialysis vol = 300 ml)
14) new process, 1x, donor unit B, 8% in DMEM (dialysis vol = 350 ml)
15) new process, 1x, donor unit C, 8% in DMEM (dialysis vol = 400 ml)
```

DATA TABLE 2
MTT assay values, normalized off blanks and 20% animal
serum control, in triplicate for each condition:

| | | | |
|---|---|---|---|
| 20% DMEM | 0.85 | 1.24 | 0.91 |
| 1 | 0.52 | 0.55 | 0.94 |
| 2 | 0.40 | 0.54 | 1.10 |
| 3 | 0.42 | 0.81 | 1.14 |
| 4 | 0.06 | 0.14 | 0.52 |
| 5 | 0.14 | 0.10 | 0.37 |
| 6 | 0.11 | 0.14 | 0.67 |
| 7 | 0.05 | 0.16 | 0.99 |
| 8 | 0.52 | 1.02 | 1.09 |
| 9 | 0.59 | 1.13 | 1.20 |
| 10 | 0.52 | 1.35 | 1.35 |
| 11 | 0.16 | 1.20 | 1.30 |
| 12 | 0.35 | 0.24 | 0.60 |
| 13 | 0.00 | 0.89 | 0.59 |
| 14 | 0.29 | 1.35 | 0.56 |
| 15 | 0.09 | 0.31 | 0.24 |

### Example 5.a

### Statistical Analysis of MTT Assay Data

One Way ANOVA done for groups:

(done with the help of the “easycalculation” online site, and using the NIST site for critical F-values, available online Jan. 5, 2014)

### Example 5.a.1

### Examining the MTT Values Resulting from '963 Process Material Applied to Three Different Units, the Null Hypothesis Hₒ=No Effective Difference in Resulting Bioactivity Across Units, can be Evaluated

1, 2, 3: F(2, 6)=0.1249, <5.143, Hₒ accepted

4, 5, 6: F(2, 6)=0.136, <5.143, Hₒ accepted

7, 8, 9: F(2, 6)=1.795, <5.143, Hₒ accepted

Thus, the '963 process appears to create a material with little inter-unit variation in the MTT assay readings.

### Example 5.a.2

### Examining the MTT Values for the Same Three Units, with Graded Application of the New Process (Dialysis Against 300, 350, or 400 ml), Pooling the 4% and 8% Groups (and Excluding the 0.0 in Group 14 as an Outlier, or Miss-Loaded Well), a Null Hypothesis Hₒ=Graded Application of New Process is not Responsible for the Observed Variation in Activity, can be Evaluated

10+13, 11+14, 12+15: F(2,14)=4.06, >3.739, so Hₒ can be rejected.

Thus, 95% confidence can be asserted that the new process is showing a real effect across these batches, assuming the effects due to dilution are small relative to those from the new process variations. Since the three new process batches were created with 40 mls in the bag, and 300, 350, or 400 mls outside, with reincorporation between the two portions, this amounts to a variation in dilution of roughly 26% (40/340=0.118, 40/390=0.103, and 40/440=0.091, so (0.118−0.091)/(average dilution=0.104)=0.259). Since these same materials were tested at two concentrations, 4%, and 8%, differences in effects due to concentration should be reflected between them, if concentration exerts a strong effect. A two-tailed t-test was done on the groups (two-tailed since no prior knowledge exists on which group can be expected to be greater). Averages for the 4 and 8% groups respectively, of 0.78556 and 0.54, yield a p-value of 0.295, giving little basis to assert a significant difference in these results. Moreover, the average change seen (a drop), with doubling of concentration across all groups, is actually opposite of what would be expected if the drop in concentration across the groups (1, 2, 3) accounts for the drop in MTT reading.

### Example 5.a.3

### Comparison of New Processes to '963 Patent Process, in Relation to Serum Values

Applying a 2-tailed, unpaired t-test on 20% serum group vs pooled 4%, '963 process group yields p=0.0004,

Thus, it can be claimed with high confidence that at a concentration of 4% the material produced using the '963 process performs significantly below standard 20% serum culture conditions.

Applying a 2-tailed, paired t-test on pooled 4%, '963 process groups vs. pooled 4% New Process groups yields a p=0.008,

Thus, it can be claimed with high confidence that the differences observed represent a significant difference resulting from the change in processes ('963 vs. the New Process) used to create these materials.

Applying a 2-tailed, unpaired t-test on the serum group vs pooled 4% New Process group yields a p=0.5, which gives little basis to claim that they are different.

Thus, the new processes bring the resulting material into the range of response of the standard serum culture conditions.

Results of these determinations in Example 5.a.3 are summarized as a graph in FIG. 1, on pg 51.

### Example 5.b

### Morphology Evidence

One picture for each culture well was collected to document the morphology of the cells at the end of the test period. A notable finding was the presence of what looked like a variable, fine precipitate, or debris on the culture surface in the serum group and groups 1-5, with conspicuous absence of this feature in groups 6-15. The possibility that this was evidence of infectious contamination was ruled out by absence of change in pH that would have been expected, and reflected in a change in media color and clarity. We interpret this finding similarly to the finding of debris and cell clumping in the skeletal muscle cell culture results, as indicating a lower quality culture environment than present in the higher numbered groups. Precipitation of protein could reflect unbalance oxidative stress generation in the media leading to denaturing effects on the proteins. Those skilled in the art would ascribe this to the presence of less healthy, metabolically robust cells in the environment that are unable to properly correct this imbalance.

Key features of these images included cells with either a fibrocyte (spindle cell) or a fibroblast character, the latter typified by 3 or more branched processes off a central mass with prominent nucleus and rough endoplasmic reticulum surrounding it. No marked change in cell number was noted by gross inspection during the culture period. Among those skilled in the art, the fibrocyte is generally considered a more quiescent form, with the fibroblast the more metabolically active, differentiated form that performs maintenance, repair and remodeling functions in tissues such as, but not limited to, the dermis, tendon and ligaments.

One skilled in the art would recognize that metabolic activity in fibroblasts may be directed to matrix synthesis, migration, or proliferation, to variable degrees, and therefore the morphological evidence should be considered together with the MTT values, to provide a holistic view of tissue remodeling responses and the relative tissue regenerative value that the observed remodeling behaviors, overall metabolic health and viability in the culture environment imply about the media supplement materials that were applied.

Evidence of organized type I collagen matrix synthesis in terms of tracks, often multiple in parallel running across the plate, was notable in some groups, likely indicative of differentiated fibroblast behavior, is in line with normal tissue remodeling behavior (Adv. Drug Del Res. 2007 Nov. 10; 59(13):1299-305. Epub 2007 Aug. 14) necessary in the dermal compartment during soft tissue wound repair, when fibroblasts first lay down a provisional matrix/ground substance, followed by organized collagen fibers. In nearly all groups we noted evidence of diffuse provisional matrix on the culture surface in terms of rolling variations in optical density, from which more metabolically and phenotypically robust cells might then be expected to develop and form organized collagen bands in line with a synergy between growth factor and matrix influences on differentiation (Adv. Drug. Del Res. 2007 Nov. 10; 59(13):1299-305. Epub 2007 Aug. 14). Computer-based optical analysis methods can be conceived by one skilled in the art for evaluation of the rolling variations in optical density observed across groups. Expression of collagen Type I is generally regarded as a key marker of fibroblast phenotype by those skilled in the art (J. Invest. Derm. 2008 April; 128(4):776-82.). Formation of aligned bands of collagen allow for active and passive tensions based on fibril interactions with ECM, which are critical to the mechanical properties of dermal tissue (Skin Res. Tech. 2003 February; 9(1):3-23), but those skilled in the art realize that the laying down of organized, aligned type I collagen is a critical aspect in the formation of tendon, ligament, and bone. In some groups we observed tracks of highly aligned bands of optically homogeneous matrix, with a thickness approximately equal to a fibroblast, migrating over the provisional matrix surface. This can be considered strong evidence to those skilled in the art, of this culture system behaving as a model of dermal compartment healing behavior at 5-6 days after injury (Int. J. Leg. Med. 1993; 106(1):31-4). It should be considered that cells aliquoted onto a culture surface can be seen, by one skilled in the art, as experiencing a simple state of injury. Subsequent behaviors can be interpreted as a “healing” response, however abbreviated the behavior might be, on a culture surface. One skilled in the art may consider the results of our work to show the extension of a standard cell culture or, “tissue culture” environment, into the range of an “organ culture” system. An ideal means of rigorously characterizing these bands of optically homogeneous matrix in the future, for regenerative applications intended to enhance dermal, tendon, or ligament healing with the products of these processes, might be to use cross-polarized light to evaluate the birefringence property of aligned Type I collagen in human fibroblast cultures like those employed here.

Ranked evaluation of the following was done from the images: 1) the relative number of “-cyte” to “-blast” forms on a total 4-star scale (distributed across “f-cytes” or “f-blasts” columns); 2) the degree of differentiation of the blast forms overall, in terms of degrees of branching, distinct nuclei and prominent rough ER, on a 2-star scale, 3) thickness of diffuse matrix, on a 3-star scale, 4) amount of aligned fibrils on a 3-star scale.

### Example 5.b.1

DATA TABLE 3
ranked morphological findings:

| | | | | diffuse | aligned |
|---|---|---|---|---|---|
| image | f-cytes | f-blasts | fb-diff | matrix | fibrils |
| 20% DMEM | +++ | + | | ++ | |
| 1 | +++ | + | + | +++ | |
| 2 | +++ | + | + | ++ | |
| 3 | ++ | ++ | ++ | ++ | |
| 4 | +++ | + | + | ++ | |
| 5 | +++ | + | + | ++ | |
| 6 | ++ | ++ | ++ | +++ | + |
| 7 | ++ | ++ | ++ | +++ | ++ |
| 8 | +++ | + | + | +++ | |
| 9 | ++ | ++ | ++ | ++ | +++ |
| 10 | ++ | ++ | + | +++ | +++ |
| 11 | ++ | ++ | ++ | ++ | ++ |
| 12 | ++ | ++ | ++ | +++ | ++ |
| 13 | + | +++ | ++ | ++ | ++ |
| 14 | ++ | ++ | ++ | ++ | + |
| 15 | ++ | ++ | + | +++ | + |

The results presented in Table 3 reveal a parallel with the MTT assay in showing an improved culture environment for human dermal fibroblasts, reflect by increased proportions of “-blast” to “-cyte” forms, increased diffuse matrix synthesis, and increased amounts of aligned fibrillar forms, as the dose of '963 process material increases (groups 1-9), and with the incorporation of new process material (groups 10-15). The best response by these metrics is shown by groups 10, 11, and 12 which (4% New Process material) and are the same groups that show the overall best responses in the MTT assay, shown in Data Table 2, and summarized in FIG. 1.

One skilled in the art could interpret these findings as reflecting the fact that regenerative bioactivities of mixtures of factors delivered to injury and clotting sites by platelets evolved to be controlled, in part, by the rate at which acidity fluctuations penetrate into spaces containing the mixtures, as well as reciprocal effects due to molecules diffusing out of these spaces, as regulated by the local conditions. The porosity and chemical affinity properties of clot structure could underlie such influences, with clear importance to regulation of angiogenesis in the stromal compartment of tumors, to one skilled in the art. The change in concentration of low molecular weight (LMW) factors, both inside and outside the bag, across the three new process variants, to a variable degree, may allow for different degrees of interaction between these LMW factors, and other elements, that can create distinct effects in the resulting mixtures, in line with Reaction-diffusion mechanisms underlying a transformation of the mixture, such as via diverse enzyme interactions with latent precursors. The exhaustive depletion, of LMW factors, may underlie major aspects of the distinct bioactivities observed with the old process. The fact that reintegration of all of these factors, but after their concentration was depleted to different degrees inside the dialysis bag for a period of time, should be considered a novel and important finding based on common understanding of such bioactivities, since no clear difference in composition would be expected, other than what was removed due to presence in the dialysis membrane. An isolated paper showed an example of a case where wound healing activity of platelets from tumor bearing animals showed an enhancement, relative to non-tumor bearing animals (Pietramaggiori, et al. (2008) Tumors Stimulate Platelet Delivery of Angiogenic Factors In Vivo—An Unexpected Benefit. December 173(6): 1609-1616), which could not be reduced to differences in angiogenic factor composition determined by SELDI-Tof, and was postulated to relate to an improved delivery of factors resulting from tumor conditioning. Our results provide one answer to how regenerative bioactivity of platelets can be transformed, that could underlie this group's observations in tumor-bearing animals. Those skilled in the art will recognize a potential similarity with the stromal compartment of tumors, which undergo dynamic interactions between clot structures and provisional matrices, which may be the influences acting on platelets through sieving properties, fluid dynamics, and affinities in the matrices, which can be seen as comparable in vivo correlates to the types of influences we postulate to underlie the bioactivity transformations observed in the work presented here.

A value of these new processes could be, in part, due to their ability to leverage an apparently natural mechanism used in the body, outside the body, for the production of activities significant to tissue regeneration, from platelets, and the ability to apply the processes in a graded fashion, to tailor these activities for greater benefits in tissue regeneration applications, than possible with prior processes. While the old process may have led to an enhancement of angiogenesis, a distinct and important wound healing activity, by permitting removal of low molecular weight elements that naturally act as angiogenesis inhibitors, the new process shows a novel benefit of reintegrating such factors into the product, to favor support of other important regenerative properties such as enhancing maintenance of desirable phenotypic characteristic of cells important in wound healing. This includes their ability to express critical matrix elements in a highly organized manner, such as production of aligned fibril bands, and basement membrane/ground substance. Regulating the differentiation of an optimal ratio of fibrocytes into the fibroblast form, enhancing cellular viability under stress, and the coincident dedication of metabolic resources necessary to support these aspects of the tissue culture environment, would be recognized by one skilled in the art as a valuable property of the resulting materials. It may also be argued that the results reported here, show that the new process can yield material able to push the boundaries of tissue culture, typical of cells in isolation, to a state approaching organ culture, as the changes observed suggest cells beginning to form a dermal organ on a culture surface.

For those skilled in the art, the presence of organized, optically homogeneous bands would be recognized for a particular importance in the regeneration of tendon and ligament tissues, which, like dermis, rely on these aligned forms for their mechanical properties. Thus, one skilled in the art would conclude that these findings are evidence of a capacity for tendon and ligament regenerative activity in the materials resulting from these new processes, and not only dermal tissue regenerative activity. Furthermore, those skilled in the art would also recognize that the mesodermal compartment of the embryo, from which dermis, tendon and ligament arise, also gives rise during development, to the blood, blood vessels, bone, cartilage, and muscle cells. The mesoderm is delineated from the ectoderm, on the outside of the embryo, that gives rise to the epidermis, and also the nervous system tissue, through it's invagination beginning along the spinal axis. The mesoderm is also delineated from the endoderm, which forms essentially all the internal organs not accounted for by ectodermal and mesodermal derivatives. An important point recognized by those skilled in the art, is that the blood and blood vessels, which are derived from mesoderm, are critical to the differentiation of all the ectodermal and endodermal organs through not only structural, and vascular connectivity functions, but also through elaboration of signals that support terminal differentiated states of these tissues and organs. Therefore, those skilled in the art would recognize that activities resulting from materials derived from platelets, which derive from the blood, that possess a capability of supporting health and beneficial remodeling properties of mesodermal derivatives, of which the fibroblast would be considered the most generic representative by those skilled in the art, can be considered to possess regenerative properties, upon delivery to any in vivo site under appropriate conditions known to those skilled in the art, that will act favorably on healing and regenerative responses of tissues of ectodermal, and endodermal origin. Those skilled in the art would recognize that this conclusion also follows from the fact that factors in platelets must have a role in all forms of healthy response to tissue injury and stress, because of their delivery to these sites through the vascular system, which would have evolved for this purpose. One skilled in the art would therefore also conclude that beneficial regenerative and tissue remodeling responses of natural factors isolated from platelets, on human fibroblasts, is evidence of beneficial regenerative activity of these materials for any organ, tissue, or cell type in the body.

While these new processes might be expected to yield material with lower angiogenic activity per unit mass, than the earlier process in the Matrigel plug assay, more “whole” mixtures, that include the total complement of factors extracted from the platelets clearly can show benefits in other aspects of wound repair, not limited to reducing the risks of applying excessive angiogenic activity, that could favor tumor formation. It might be considered that in the dialysis bag, which is removed from the final product, a portion of factors enriched in the MW range of the membrane (6-8 kd for these experiments), will be removed with the bag. Thus, the choice of MW of the membrane also holds a means to deplete a portion of elements over a distinct MW range, with only a single membrane process. The relative volume ratio of the membrane, to the total contained, and external volumes would have to be considered in gauging the overall percent depletion. Subsequent rinsing to various degrees, or digestion of the membrane, to capture, or determine contents from the platelet extracts remaining, would be important in making such a separation approach effective, presuming different protein affinities for dialysis membrane materials.

These results are counterintuitive, based on standard views to the role of angiogenesis in tissue regeneration, in which the healing benefits of the first patent process, observed in mouse incision wounds, would be considered a result of angiogenic activity, and reflected in capillary formation in Matrigel plugs. A reasonable hypothesis one skilled in the art might reach regarding the enhancement in activity observed for the first patent (963) is that low molecular weight elements in the mixture, lost from the bag, have an anti-angiogenic activity. Based on this reasoning, return of these factors should reduce the angiogenic, and hence tissue regenerative activity of the mixture as measured in the Matrigel plug assay. Tests on human cells were chosen instead for the possibility of demonstrating tissue regenerative and morphogenic activity, not strictly referable to capillary sprouting activity. One skilled in the art might conclude that more value exists in mixtures of factors from platelets that can show beneficial influences on subtle tissue remodeling processes shown here, than for an ability to initiate sprouting and stabilization of new blood vessel structures in vivo, as would be reflected in capillary counts in Matrigel plugs implanted in mice.

The morphological and MTT evidence suggest that the new process can yield a more potent material than the old process (less mass needed for similar effect), and therefore that it may have a better therapeutic index, than products of the '963 patent, in the view of those skilled in the art. These results would also suggest to one skilled in the art that the new process materials will have superior ability to favor cellular viability and phenotypic properties of human dermal fibroblasts beneficial to soft tissue wound healing, typified, but not restricted to, settings such as laser-resurfacing wound treatment, injections for tendinopathy such as tennis elbow (lateral epichondylitis), or any treatment which seeks to enhance healing, or provide a therapeutic influence to a mesenchymal tissue such as bone, cartilage, ligament, or muscle, which are generally considered to result from differentiated forms of the fibroblast lineage. Those skilled in the art would agree that these cell types may be brought, in natural circumstances, into tissues for regenerative and remodeling purposes through the blood, and can be derived from a mononuclear lineage, that can transit through a fibroblast cell-type in the course of differentiation toward the more highly specialized forms listed above. The evidence presented here shows enhanced formation by human fibroblasts of organized aligned, optically homogeneous, matrix structures, which are critical for regeneration of soft tissue injuries of the dermis, tendons and ligaments. Among those skilled in the art, it would be strongly suspected that these processes yield materials with improved cartilage, bone, and muscle tissue regenerative activity relative to the '963 patent material.

Those skilled in the art would agree that these benefits could, in part, be attributed to a “whole food,” type property of the material resulting from the new processes, perhaps due to a larger portion of the entire complement of factors naturally present in the platelets remaining in the product, rather than predominantly a fraction above a particular molecular weight, characteristic of the '963 patent product.

It may be wondered how something as simple as acidity (proton concentration), or any small molecule with rapid diffusion capability, act as a control factor of powerful regenerative bioactivity? Those skilled in the art typically recognize major morphogens as large protein molecules like BMPs/TGFbetas, FGFs, Hedgehogs, etc.

These inventions have been informed by the Theory of De Broglie, now solidly experimentally established as an aspect of quantum mechanics, whereby a proton, or any particle, also has properties of a wave (and it's not “either/or,” but rather a matter of which aspect is most evident in a given setting.) Under this theory, de Broglie's relations yield wavelength as inversely proportional to momentum (mv) for velocities<<c (the speed of light) a good assumption for protons in a tissue with many negatively charged groups present in the hydrated media. These conditions would also imply propagation through a dispersive media that requires a consideration of phase and group velocity for a more complete description, which will not be developed here. Those skilled in the art would agree that an estimate of a low proton momentum in a dispersive environment rich with negatively charged groups, such as most tissues, would yield the longest effective “matter wave,” of any freely diffusing element, among those widely known in biological systems. This means a change in [H+] at one site in an organism, could be sense more rapidly, than the change in any other molecule's concentration, at a site of any particular distance from this disturbance. Cells are widely considered to be very sensitive to pH, by those skilled in the art.

This work has been informed by the idea that it would be unlikely that this mode of communication across a field of cells, with it's inherent simplicity, would have been overlooked by evolutionary pressures. Protein conformation is sensitive to pH, and so a wave of [H+] shift could have the more rapid influence, across an entire cell, on all of it's receptor affinities for ligands, than other lumbering morphogenic factors of higher MW, thereby providing a basis for the power of proton waves to influence important changes in cells across entire tissues, essentially, all-at-once, based on the wave-like behavior of protons.

1. A composition comprising a platelet extract comprising a platelet-derived growth factor and a part, but not all, of a protein dialysate.

2. A process for producing a platelet extract having an angiogenic or vascular normalizing activity and a dermal, tendon and ligament tissue regenerative activity comprising
a. disrupting pelletized platelets in an extraction solution to produce a resulting extraction solution comprising an insoluble material, a growth factor, and a protein capable of separation from the growth factor based on difference in their Stokes radius;
b. exhaustively depleting in the resulting extraction solution the concentration of the insoluble material and the protein relative to the concentration of the growth factor; and
c. reintroducing a portion of the protein into the resulting extraction solution to produce a platelet extract having an activity selected from the group consisting of an angiogenic activity, a vascular normalizing activity, an ectoderm-derived tissue regenerative activity, an endoderm-derived tissue regenerative activity, a mesoderm-derived tissue regenerative activity, and a dermal, tendon and ligament tissue regenerative activity.

3. The process of claim 2, wherein the reintroducing of step c. is replaced by reducing, but non-exhaustively depleting, the protein in step b.
